(19) United States
(12) Patent Application Publication
(10) Pub. No.: US 2013/0078655 A1
(43) Pub. Date: Mar. 28, 2013

(54) KIDNEY PROGNOSTIC ASSAY

(71) Applicant: Arthur R. Bradwell, Birmingham (GB)

(72) Inventor: Arthur R. Bradwell, Birmingham (GB)

(73) Assignee: THE BINDING SITE GROUP LIMITED, Birmingham (GB)

(21) Appl. No.: 13/581,883

(22) PCT Filed: Mar. 3, 2011
(86) PCT No.: PCT/IB2011/050919

§ 371 Date: Nov. 28, 2012

(30) Foreign Application Priority Data
Mar. 2, 2010 (GB) 1003485.8

(51) Int. Cl. G01N 33/566; G01N 21/76; G01N 21/64

(52) U.S. Cl. 435/7.94; 436/501

(57) ABSTRACT

The invention provides a method of predicting subjects at risk of loss of kidney function and/or identifying subjects at greater risk of loss of kidney function, and/or identifying subjects at risk of kidney failure/end stage kidney disease, the method comprising detecting an amount of free light chains (FLC) in a sample from the subject, wherein a higher amount of FLC is associated with increased risk of loss of kidney function and/or increased risk of renal failure/end stage kidney disease. A further aspect of the invention provides a method of monitoring renal impairment, comprising detecting an amount of free light chains (FLC) in a sample from a subject having renal impairment and comparing the amount of FLC in the sample with an Total FLC amount of FLC detected in a sample previously obtained from the subject, wherein an increase in the amount FLC detected, compared to the previous sample, indicates an increase in the risk of loss of renal function in the subject, and a decrease in the amount of FLC indicates a decrease in the risk of loss of renal function in the subject.

The invention relates to a method of predicting patients at risk of loss of kidney function, identifying a subject at greater risk of loss of kidney function and/or identifying a subject at risk of renal failure or end stage kidney disease

The Applicants have for many years studied free light chains as a way of assaying for a wide-range of monoclonal gammopathies in patients. The use of such free light chains in diagnosis is reviewed in detail in the book “Serum Free Light Chain Analysis, Fifth Edition (2008) A. R. Bradwell et al, ISBN 0704427028”.

Antibodies comprise heavy chains and light chains. They usually have a two-fold symmetry and are composed of two identical heavy chains and two identical light chains, each containing variable and constant region domains. The variable domains of each light-chain/heavy-chain pair combine to form an antigen-binding site, so that both chains contribute to the antigen-binding specificity of the antibody molecule. Light chains are of two types, κ and λ and any given antibody molecule is produced with either light chain but never both. There are approximately twice as many κ as λ molecules produced in humans, but this is different in some mammals. Usually the light chains are attached to heavy chains. However, some unattached “free light chains” are detectable in the serum or urine of individuals. Free light chains may be specifically identified by raising antibodies against the surface of the free light chain that is normally hidden by the binding of the light chain to the heavy chain. In free light chains (FLC) this surface is exposed, allowing it to be detected immunologically. Commercially available kits for the detection of κ or λ free light chains include, for example, “Freelite™”, manufactured by The Binding Site Limited, Birmingham, United Kingdom. The Applicants have previously identified that determining free κ/free λ ratios, aids the diagnosis of monoclonal gammopathies in patients. It has been used, for example, as an aid in the diagnosis of intact immunoglobulin multiple myeloma (MM), light chain MM, non-secretory MM, AL amyloidosis, light chain deposition disease, smouldering MM, plasmacytoma and MGUS (monoclonal gammopathies of undetermined significance). Detection of FLC has also been used, for example, as an aid to the diagnosis of other B-cell dyscrasia and indeed as an alternative to urinary Bence Jones protein analysis for the diagnosis of monoclonal gammopathies in general.

Conventionally, an increase in one of the λ or κ light chains and a consequently abnormal ratio is looked for. For example, multiple myelomas result from the monoclonal multiplication of a malignant plasma cell, resulting in an increase in a single type of cell producing a single type of immunoglobulin. This results in an increase in the amount of free light chain, either λ or κ, observed within an individual. This increase in concentration may be determined, and usually the ratio of the free κ to free λ is determined and compared with the normal range. This aids in the diagnosis of monoclonal disease. Moreover, the free light chain assays may also be used for the following of treatment of the disease in patients. Prognosis of, for example, patients after treatment for AL amyloidosis may be carried out.

Katzman et at (Clin. Chem. (2002); 48(9): 1437-1944) discuss serum reference intervals and diagnostic ranges for free κ and free λ immunoglobulins in the diagnosis of monoclonal gammopathies. Individuals from 21-90 years of age were studied by immunoassay and compared to results obtained by immuno fixation to optimise the immunoassay for the detection of monoclonal free light chains (FLC) in individuals with B-cell dyscrasia. The amount of κ and λ FLC and the κ/λ ratios were recorded allowing a reference interval to be determined for the detection of B-cell dyscrasias.

Renal failure is a major cause of morbidity and mortality in patients with multiple myeloma (MM). At initial presentation with MM up to 50% of patients have renal impairment, 12 to 20% have acute renal failure and 10% become dialysis dependent. This represents about 2% of the dialysis population (Bradwell A. R. Serum Free Light Chain Analysis, 5^{th} Edn, The Binding Site Ltd 2008). Monoclonal FLCs are one of the most potent causes of irreversible renal failure. FLC can, for example, physically block tubules.

Monoclonal FLCs cause renal failure by several different mechanisms, any of which may contribute to both acute myeloma kidney and chronic renal failure. In MM, monoclonal sFLC can have a wide range of concentrations. Moreover, their toxicity has previously been shown to vary considerably.

Concentrations of sFLC (serum Free Light Chains) necessary to cause renal impairment in MM have been studied. Additionally, urine FLC excretion rates have also been studied. FLC excretion was found to be an indicator of renal damage in addition to its cause.

Studies have shown that sFLC κ/λ ratios are a simple method for identifying monoclonal FLC production in patients with MM and acute renal failure.

However, no work has been carried out to correlate concentrations of sFLC with the risk of renal failure in non-MM patients.

The Applicant has now identified a correlation between total FLCs and the risk of developing progressive renal failure in individuals without MM or associated conditions. This is distinct to acute renal failure observed which sometimes occurs in MM. The physical blocking of the tubules in MM patients typically occurs at >500 mg/L FLC in blood.

The Applicants have found that, for example, FLCs in chronic kidney disease (typically 50-200 mg/L) may be markers for predicting the risk of an increased decline in renal function. Polyclonal FLCs at these concentrations have not previously been reported to cause significant damage to the kidneys, but are markers of reduced glomerular filtration and any increased inflammation.

The concentration of polyclonal FLCs in serum from individuals that are apparently healthy is influenced by the rate of production and the rate of removal; determined by the ability of the individual's kidneys to filter FLC. In individuals where FLC clearance is restricted, there is an increase in the levels of FLC found in serum. As a consequence, it is now believed that FLC is a marker of renal function. Because monomeric FLC kappa molecules (25 kDa) and dimeric lambda molecules (50 kDa), are significantly different sized molecules to creatinine 113 Da together they offer an alternative marker of glomerular filtration). However, in contrast to creatinine, increased production of FLCs may result as a consequence of many diseases, so serum FLCs will typically not be used as a renal function marker, in isolation.

However, markers of B-cell proliferation/activity are important and because B-cells are responsible for making FLCs, this is clinically useful. FLC production is an early indicator of B-cell up-regulation. In this respect it can complement the use of CRP which is a T-cell mediated marker of inflammatory responses.

High FLC concentrations are an indication of chronic renal or inflammatory disorders or B-cell dyscrasias. Hence, an abnormal FLC assay result may be a marker of a variety of disorders that currently require several diagnostic tests in combination. The converse of this, when the FLC assay results are normal, indicates good renal function, no inflammatory conditions and no evidence of B-cell dyscrasia.

The invention provides a method of predicting subjects at risk of loss of kidney function and/or identifying subjects at greater risk of loss of kidney function, and/or identifying subjects at risk of kidney failure/end stage kidney disease, the method comprising detecting an amount of free light chains (FLC) in a sample from the subject, wherein a higher amount of FLC is associated with increased risk of loss of kidney function and/or increased risk of renal failure/end stage kidney disease.

The subject may be apparently healthy or have indications of renal impairment, such as chronic kidney disease (CKD).

A further aspect of the invention provides a method of prognosis of a subject with renal impairment comprising detecting an amount of FLC in a sample from the subject, wherein a higher amount of FLC is associated with increased risk of loss of renal function.

A further aspect of the invention provides a method of monitoring renal impairment, comprising detecting an amount of free light chains (FLC) in a sample from a patient having renal impairment and comparing the amount of FLC in the sample with an amount of FLC detected in a sample previously obtained from the patient, wherein an increase in the amount FLC detected, compared to the previous sample, indicates an increase in the risk of loss of renal function in the patient, and a decrease in the amount of FLC indicates a decrease in the risk of loss of renal function in the patient. This may be used, for example, to monitor the effectiveness of a treatment, such as an antihypertensive or immunosuppressant.

The FLC may be kappa or lambda FLC. However, preferably the total FLC concentration (lambda and kappa FLC) is measured, as detecting kappa FLC or lambda FLC alone may miss, for example, abnormally high levels of one or other FLC produced monoclonally in the patient.

Total free light chain means the total amount of free kappa plus free lambda light chains in a sample.

Preferably the subject does not necessarily have symptoms of a B-cell associated disease. The symptoms may include recurrent infections, bone pain and fatigue. Such a B-cell associated disease is preferably not a monoclonal FLC disease. Typically it is not a myeloma, (such as intact immunoglobulin myeloma, light chain myeloma, non-secretory myeloma), an MGUS, AL amyloidosis, Waldenström's macroglobulinaemia, Hodgkin's lymphoma, follicular centre cell lymphoma, chronic lymphocytic leukaemia, mantle cell lymphoma, pre-B cell leukaemia or acute lymphoblastic leukaemia. Moreover, the individual typically does not have reduced bone marrow function. The individual typically does not have an abnormal κ:λ FLC ratio, typically found in many such diseases.

The sample is typically a sample of serum from the subject. However, whole blood, plasma, urine or other samples of tissue or fluids may also potentially be utilised.

Typically the FLC, such as total FLC, is determined by immunoassay, such as ELISA assays or utilising fluorescently labelled beads, such as Luminex™ beads.

Sandwich assays, for example use antibodies to detect specific antigens. One or more of the antibodies used in the assay may be labelled with an enzyme capable of converting a substrate into a detectable analyte. Such enzymes include horseradish peroxidase, alkaline phosphatase and other enzymes known in the art. Alternatively, other detectable tags or labels may be used instead of, or together with, the enzymes. These include radioisotopes, a wide range of coloured and fluorescent labels known in the art, including fluorescein, Alexa fluor, Oregon Green, BODIPY, rhodamine red, Cascade Blue, Marina Blue, Pacific Blue, Cascade Yellow, gold; and conjugates such as biotin (available from, for example, Invitrogen Ltd, United Kingdom). Dye sols, chemiluminescent labels, metallic sols or coloured latex may also be used. One or more of these labels may be used in the ELISA assays according to the various inventions described herein or alternatively in the other assays, labelled antibodies or kits described herein.

The construction of sandwich-type assays is itself well known in the art. For example, a “capture antibody” specific for the FLC is immobilised on a substrate. The “capture antibody” may be immobilised onto the substrate by methods which are well known in the art. FLC in the sample are bound by the “capture antibody” which binds the FLC to the substrate via the “capture antibody”.

Unbound immunoglobulins may be washed away.

In ELISA or sandwich assays the presence of bound immunoglobulins may be determined by using a labeled “detecting antibody” specific to a different part of the FLC of interest than the binding antibody.

Flow cytometry may be used to detect the binding of the FLC of interest. This technique is well known in the art for, e.g. cell sorting. However, it can also be used to detect labeled particles, such as beads, and to measure their size. Numerous text books describe flow cytometry, such as Practical Flow Cytometry, 3rd Ed. (1994), H. Shapiro, Alan R. Liss, New York, and Flow Cytometry, First Principles (2nd Ed.) 2001, A. L. Given, Wiley Liss.

One of the binding antibodies, such as the antibody specific for FLC, is bound to a bead, such as a polystyrene or latex bead. The beads are mixed with the sample and the second detecting antibody. The detecting antibody is preferably labeled with a detectable label, which binds the FLC to be detected in the sample. This results in a labeled bead when the FLC to be assayed is present.

Other antibodies specific for other analytes described herein may also be used to allow the detection of those analytes.

Labeled beads may then be detected via flow cytometry. Different labels, such as different fluorescent labels may be used for, for example, the anti-free λ and anti-free κ antibodies. Other antibodies specific for other analytes, such as bacterial-specific antigens, described herein may also be used in this or other assays described herein to allow the detection of those analytes. This allows the amount of each type of FLC bound to be determined simultaneously or the presence of other analytes to be determined.

Alternatively, or additionally, different sized beads may be used for different antibodies, for example for different marker specific antibodies. Flow cytometry can distinguish between different sized beads and hence can rapidly determine the amount of each FLC or other analyte in a sample.

An alternative method uses the antibodies bound to, for example, fluorescently labeled beads such as commercially available Luminex™ beads. Different beads are used with different antibodies. Different beads are labeled with different fluorophore mixtures, thus allowing different analytes to be determined by the fluorescent wavelength. Luminex beads are available from Luminex Corporation, Austin, Tex., United States of America.

Preferably the assay used is a nephelometric or turbidimetric method. Nephelometric and turbidimetric assays for the detection of λ- or κ-FLC are generally known in the art, but not for total FLC assays. They have the best level of sensitivity for the assay. λ and κ FLC concentrations may be separately determined or a single assay for total FLC arrived at. Such an assay contains anti-κ and anti-λ FLC antibodies typically at a 60:40 ratio, but other ratios, such as 50:50 may be used.

Antibodies may also be raised against a mixture of free λ and free κ light chains.

The amount of total FLC may be compared to a standard, predetermined value to determine whether the total amount is higher or lower than a normal value.

As discussed in detail below, the Applicants have identified that higher concentrations of serum FLC are associated with a significant increase in the likelihood of loss of renal function in patients. More so than, for example, for people with lower serum FLC levels.

An absolute level of >68 mg/L of FLCs or a corrected level of >1.7 mg/L of FLC per unit GFR was associated with an increased risk of loss of kidney function or renal failure.

Historically, assay kits have been produced for measurement of kappa and lambda FLC separately, to allow the calculation of a ratio. They have been conventionally used in individuals already exhibiting disease symptoms.

Preferably the assay is capable of determining FLC, for example total FLC, in the sample for example from approximately 1 mg/L to 100 mg/L, or 1 mg/L-80 mg/L. This is expected to detect the serum FLC concentrations in the vast majority of individuals without the requirement for re-assaying samples at a different dilution.

Preferably the method comprises detecting the amount of total free light chain in the sample utilising an immunoassay, for example, by utilising a mixture of anti-free κ light chain and anti-free λ light chain antibodies or fragments thereof. Such antibodies may be in a ratio of 50:50 anti-κ: anti-λ antibodies. Antibodies, or fragments, bound to FLC may be detected directly by using labelled antibodies or fragments, or indirectly using labelled antibodies against the anti-free λ or anti-free κ antibodies.

The antibodies may be polyclonal or monoclonal. Polyclonal may be used because they allow for some variability between light chains of the same type to be detected as they are raised against different parts of the same chain. The production of polyclonal antibodies is described, for example in WO97/17372.

Preferably, the amount of serum FLC, such as total FLC, identified, and found to be significant to show an increased likelihood of loss of kidney function is at least 68 mg/L or at least 1.7 mg/L FLC per unit GFR.

Assay kits for FLC, for example for use in the methods of the invention are also provided. The kits may detect the amount of total FLC in a sample. They may be provided in combination with instructions for use in the methods of the invention.

Assay kits are also for use in a method according to the invention, comprising one or more anti-FLC antibodies and one or more reagents for the detection of other markers of kidney function, such as creatinine, urea or cystatin C and/or reagents for the assay of urinary markers of kidney function such as albumin or urinary free light chains.

The assay kits may be adapted to detect an amount of total free light chain (FLC) in a sample below 25 mg/L, most preferably, below 20 mg/L or about, 10 mg/L, below 5 mg/L or 4 mg/L. The calibrator material typically measures the range 1-100 mg/L. The assay kit may be, for example, a nephelometric assay kit. Preferably the kit is an immunoassay kit comprising one or more antibodies against FLC. Typically the kit comprises a mixture of anti-κλand anti-λ FLC antibodies. Typically a mixture of 50:50 anti-free κ and anti-free λ antibodies are used. The kit may be adapted to detect an amount of 1-100 mg/L, or preferably 1-80 mg/L total free light chain in a sample.

Fragments of antibodies, such as (Fab)₂ or Fab antibodies, which are capable of binding FLC may also be used.

The antibodies or fragments may be labelled, for example with a label as described above. Labelled anti-immunoglobulin binding antibodies or fragments thereof may be provided to detect anti-free λ or anti-free κ bound to FLC.

The kit may comprise calibrator fluids to allow the assay to be calibrated at the ranges indicated. The calibrator fluids preferably contain predetermined concentrations of FLC, for example 100mg/L to 1 mg/L, below 25 mg/L, below 20 mg/L, below 10 mg/L, below 5 mg/L or to 1 mg/L. The kit may also be adapted by optimising the amount of antibody and “blocking” protein coated onto the latex particles and, for example, by optimising concentrations of supplementary reagents such as polyethylene glycol (PEG) concentrations.

The kit may comprise, for example, a plurality of standard controls for the FLC. The standard controls may be used to validate a standard curve for the concentrations of the FLC or other components to be produced. Such standard controls confirm that the previously calibrated standard curves are valid for the reagents and conditions being used. They are typically used at substantially the same time as the assays of samples from subjects. The standards may comprise one or more standards below 20 mg/L for FLC, more preferably below 15 mg/L, below approximately 10 mg/L or below 5 mg/L, in order to allow the assay to calibrate the lower concentrations of free light chain.

The assay kit may be a nephelometric or turbidimetric kit. It may be an ELISA, flow cytometry, fluorescent, chemiluminescent or bead-type assay or dipstick. Such assays are generally known in the art.

The assay kit may also comprise instructions to be used in the method according to the invention. The instructions may comprise an indication of the concentration of total free light chain considered to be a normal value, below which, or indeed above which, shows an indication of either increased or decreased probability of loss of kidney function for the individual, for example. Such concentrations may be as defined above.

The invention will now be described by way of example only, with reference to the following figures:

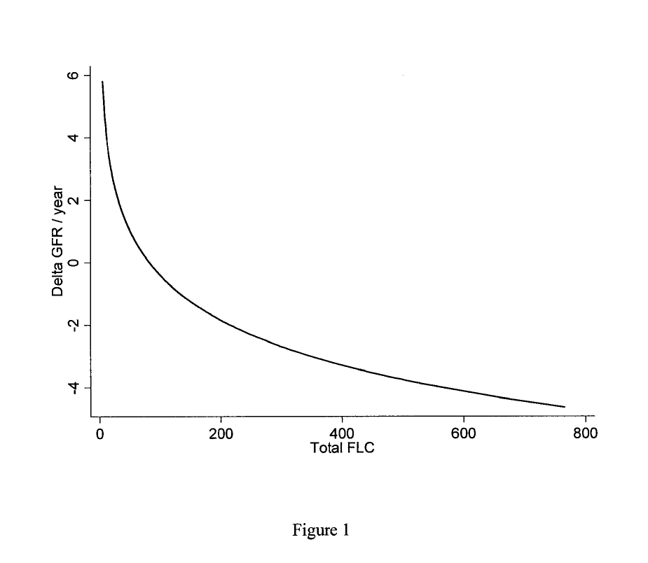

FIG. 1 shows the change of renal function (delta GFR) compared to total FLC concentration in serum (mg/L).

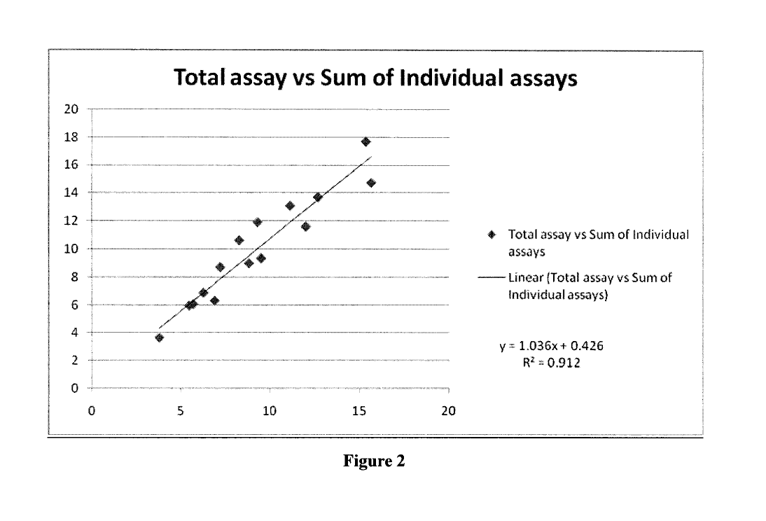

FIG. 2 is a comparison between the total FLC concentrations obtained using separate, commercially available, anti-free κ and anti-free λ assay kits, compared to a total FLC assay kit using combined anti-λ and anti-κ free light chain antibodies.

### Renal Function Prognosis

### Methods

1300 patients with various degrees of renal impairment had serum samples collected (“Baseline”) and were then followed up for a period of up to 63 months.

In more detail, the patients were recruited from the renal clinics at the University Hospital Birmingham. The patients had a range of renal problems including reduced GFR, proteinuria, haematuria, chronic kidney disease (all stages), end stage renal failure (haemodialysis and peritoneal dialysis) and renal transplant recipients.

The tests and assessments made included:
Serum creatinine and an estimated glomerular filtration rate (eGFR).
A corrected level of FLCs per unit GFR was calculated as follows: total serum FLC concentration (mg/L) was divided by estimated glomerular filtration rate as calculated by the Cockcroft-Gault equation (REF) in mls/min/1.73 m2. Thus giving a serum total FLC level for the patient, independent of renal function, in mg/L per unit GFR.
Ref:
Cockcroft D W, Gault M H: Prediction of creatinine clearance from serum creatinine. Nephron 16: 31-41, 1976.
Urinary albumin/creatinine ratio.
Serum FLC concentrations, both kappa and lambda (Freelite, The Binding Site, Birmingham, UK).
Total, serum FLC concentrations were calculated by adding the values for kappa FLC and lambda FLC.

### Follow-up:

Patients were followed up for rate of decline of renal function.

### Results

During follow-up the risk of progression (loss of renal function or delta GFR) was strongly related to the total serum FLC concentrations:

| | | dGFR | |
|---|---|---|---|
| FLC quintile | 1st quintile | 0 | P = 0.003 |
| | 2nd quintile | −1.1 (−3.2, 1.1) | |
| | 3rd quintile | −2.7 (−4.8, −0.9) | |
| | 4th quintile | −3.7 (−5.8, −1.6) | |
| | 5th quintile | −3.2 (−5.7, −0.9) | |

With higher total FLC levels being associated with a greater loss of eGFR as shown in FIG. 1.

In a multivariant analysis the only factors which independently predicted loss of renal function were patient age and total FLC. Briefly delta GFR was measured on a continuous scale, and an examination of the distribution of the values suggested that these were normally distributed. Therefore, linear regression was used for the analysis. Initially the individual effect of each predictor variable upon recovery was examined (univariable analysis). Subsequently the joint effect of the explanatory variables upon each outcome was examined in a multivariable analysis. An advantage of this method is that the effect of each explanatory variable upon the outcome is adjusted for the effects of all other explanatory variables in the regression model. Therefore, this gives a more accurate picture of the variables which have an underlying effect upon the outcome, and not just those which might be reflecting the effects of other variables. A backwards selection procedure was used to retain only those variables that were statistically significant. This involves removing non-significant variables from the model one at a time, until all remaining variables are statistically significant. Only variables showing some effect upon the outcome from the univariable analyses (p<0.2) were considered for the multivariable analyses.

| Variable | Category | Coefficient (95% CI) | P-value |
|---|---|---|---|
| Age ⁽*⁾ | — | −0.7 (−1.2, −0.3) | <0.001 |
| Total FLC ^{(#)} | — | −1.4 (−2.6, −0.2) | 0.02 |

⁽*⁾ Coefficients given for a10 unit increase in explanatory variable
^{(#)} Variable analysed on the log scale

Total FLCs independently predict loss of renal function.

### Discussion

The results demonstrate that total FLCs independently identify patients at risk of developing progressive renal failure. This provides clinicians with a completely novel risk stratification tool for the management of patients with renal impairment. This is potentially most relevant to general practitioners who are charged with the task of looking after a growing population of patients who are identified as having CKD. Total serum FLC concentration provides them with a novel tool for risk stratifying this population.

### Assay Kit

The method according to the invention may utilise the following assay kit. The assay kit quantifies the total free κ plus free λ light chains present within patient samples, for example, in serum. This may be achieved by coating 100 nm carboxyl modified latex particles with a 50:50 blend of anti-free κ and anti-free λ light chain sheep antibody. In the assay exemplified below, the measuring range for the total free light chains is for 1-80 mg/L. However, other measuring ranges could equally be considered.

Anti-free κ and anti-free λ anti sera are produced using techniques generally known in the art, in this particular case in sheep. The general immunisation process is described in WO 97/17372.

Anti-κ and anti-λ antisera were diluted to equal concentrations using phosphate buffered saline (PBS). Those antibodies were combined to produce antisera comprising 50% anti κ antibody and 50% anti λ antibody.

Antibodies were coated onto carboxyl modified latex at a coat load of 10 mg/lot. This was achieved using standard procedures. See, for example, “Microparticle Reagent Optimization: A laboratory reference manual from the authority on microparticles” Eds: Caryl Griffin, Jim Sutor, Bruce Shull. Copyright Seradyn Inc, 1994 (P/N 0347835(1294).

This reference also provides details of optimising the assay kits using polyethylene glycol (PEG).

The combined antibodies were compared to results obtained using commercially available κ and λ Freelite™ kits (obtained from the Binding Site Group Limited, Birmingham, United Kingdom). Such Freelite™ kits identify the amount of κ and the amount of λ free light chains in separate assays. The total FLC kits were used to generate curves, which were validated using controlled concentrations. Calibration curves were able to be obtained between 1 and 80 mg/l for total free light chain. In the results table below, results were obtained for κ free light chain (KFLC), κ free light chain (LFLC) and total FLC, using the κ Freelite™, λ Freelite™ and total free light chain assays. These results are shown for 15 different normal serum samples. The results are shown in the table below and in FIG. 2 as measured by turbidimetry.

Preliminary results indicate that the principle of using a total free light chain assay based on anti-κ and anti-λ free light chain antibody is viable.

Results

| | | | | | | % diff Total FLC |
|---|---|---|---|---|---|---|
| | Batch | Results (mg/l) | | | KFLC + | vs (KFLC + |
| USN | Id | KFLC | LFLC | Total FLC | LFLC | LFLC) |
| 1 | 104 | 3.37 | 3.51 | 6.31 | 6.88 | −8.3% |
| 2 | 151 | 3.42 | 5.39 | 8.99 | 8.81 | 2.0% |
| 3 | 158 | 3.28 | 6.21 | 9.35 | 9.49 | −1.5% |
| 4 | 161 | 2.05 | 3.62 | 6.06 | 5.67 | 6.9% |
| 5 | 179 | 6.83 | 5.84 | 13.71 | 12.67 | 8.2% |
| 6 | 180 | 2.19 | 3.27 | 5.96 | 5.46 | 9.2% |
| 7 | 181 | 2.98 | 5.27 | 10.64 | 8.25 | 29.0% |
| 8 | 182 | 4.72 | 7.26 | 11.6 | 11.98 | −3.2% |
| 9 | 216 | 2.54 | 4.66 | 8.7 | 7.2 | 20.8% |
| 10 | 217 | 3.01 | 3.24 | 6.88 | 6.25 | 10.1% |
| 11 | 219 | 7.12 | 8.53 | 14.73 | 15.65 | −5.9% |
| 12 | 227 | 1.47 | 2.31 | 3.66 | 3.78 | −3.2% |
| 13 | 228 | 8.16 | 7.2 | 17.67 | 15.36 | 15.0% |
| 14 | 229 | 4.51 | 6.61 | 13.1 | 11.12 | 17.8% |
| 15 | 231 | 3.69 | 5.6 | 11.91 | 9.29 | 28.2% |
| | | | | | Mean | 8.3% |
| | | | | | Diff | |

What is claimed is:

1. A method of predicting subjects at risk of loss of kidney function and/or identifying subjects at greater risk of loss of kidney function, and/or identifying subjects at risk of kidney failure/end stage kidney disease, the method comprising detecting an amount of free light chains (FLC) in a sample from the subject, wherein a higher amount of FLC is associated with increased risk of loss of kidney function and/or increased risk of renal failure/end stage kidney disease.

2. A method of monitoring renal impairment, comprising detecting an amount of free light chains (FLC) in a sample from a subject having renal impairment and comparing the amount of FLC in the sample with an amount of FLC detected in a sample previously obtained from the subject, wherein an increase in the amount FLC detected, compared to the previous sample, indicates an increase in the risk of loss of renal function in the subject, and a decrease in the amount of FLC indicates a decrease in the risk of loss of renal function in the subject.

3. A method according to claim 1, wherein the subject does not exhibit symptoms of a B-cell associated disease.

4. A method according to claim 1, wherein the subject does not exhibit symptoms of multiple myeloma.

5. A method according to claim 1, wherein the amount of free light chains is the amount of total free light chains in the sample.

6. A method according to claim 5, wherein the FLC is determined in a sample of serum from the subject.

7. A method according to claim 6, wherein the total FLC is determined by immunoassay using anti-free light chain antibodies.

8. A method according to claim 7, wherein the antibodies are a mixture of anti-free K light chain and anti-free λ light chain antibodies.

9. A method according to claim 8, wherein the method comprises detecting the amount of FLC by nephelometry or turbidimetry.

10. (canceled)

11. An assay kit for use in the method according to claim 1, said kit comprising
one or more anti-FLC antibodies;
a normal value against which a concentration of FLC obtained using the assay kit, indicates an increased survival of a subject, when the value exceeds the normal value.

12. An assay kit for use in a method according to claim 11, said kit further comprising
one or more assays for other markers of renal function (such as serum creatinine, urea or cystatin C) and/or reagents for the assay of urinary markers of kidney function (such as albumin or urinary free light chains).

13. An assay kit for use in a method according to claim 12, said kit further comprising a set of instructional materials.

14. A method of prognosing a subject at risk of loss of kidney function, said method comprising
analyzing a sample isolated from said subject to measure the amount of free light chains (FLC) present in said sample;
determining if the detected concentration of free light chains (FLC) present in said sample exceeds a normal value, wherein an amount of FLC higher than the normal value is associated with an increased risk of loss of kidney function in said subject.
